(19) United States
(12) Patent Application Publication
(10) Pub. No.: US 2007/0212775 A1
(43) Pub. Date: Sep. 13, 2007

(54) MICROTITER PLATE, METHOD OF MANUFACTURING THEREOF AND KIT

(75) Inventors: David A. Cohen, Dedham, MA (US); Michael J. Mortillaro, Webster, NY (US); Bruce R. Turner, Exeter, NH (US)

Correspondence Address: BIRCH STEWART KOLASCH & BIRCH, PO BOX 747, FALLS CHURCH, VA, 22040-0747, US

(73) Assignee: FINNZYMES INSTRUMENTS OY, Espoo (FI)

(21) Appl. No.: 11/623,025

(22) Filed: Jan. 12, 2007

Related U.S. Application Data
(60) Provisional application No. 60/758,775, filed on Jan. 13, 2006.

(51) Int. Cl. C12M 1/34; B01L 3/00

(52) U.S. Cl. 435/288.4; 435/287.2; 435/299.2; 435/303.1; 435/809; 422/102

(57) ABSTRACT

The invention relates to a vessel and kit for thermal cycling applications, a method for manufacturing such a vessel. The vessel comprises, in the form of a planar grid having a predefined pitch, a plurality of sample wells each having a well wall, which defines an open well end and a closed well end. According to the invention there are provided a plurality of ribs between pairs of adjacent wells, the ribs being connected to the walls of the wells and extending essentially in a plane perpendicular to the plane of the well grid. The invention enables manufacturing of dense microtiter plates, which are stable enough to be used in high temperature applications and allow for more efficient manufacturing of the plate.

### BACKGROUND OF THE INVENTION

1. Field of the Invention

This application claims benefit of priority under 35 U.S.C. 119(e) to U.S. Provisional Application No. 60/758,775, filed Jan. 13, 2006, the entirety of which is incorporated herein by reference.

The invention relates to processing of biological samples. In particular, the invention concerns microtiter plates, which are commonly used for performing Polymerase Chain Reaction (PCR) Processes. Such plates have a plurality of wells arranged in a grid, each of the wells being capable of holding a small amount of biological sample. The invention concerns also a method of manufacturing such plates, use of such a method and a kit including such plates.

2. Description of Related Art

Biological samples are processed in industrial and clinical diagnostics, pharmaceutical and research applications, and as processes have improved, the need for increasing the number and speed of samples processed has also increased.

The standards that are most commonly used are based on the formats of the microfuge tube, the microscope slide and the microtiter plate. Microfuge tubes come in several, usually non-interchangeable sizes based on the desired volume of the sample to be processed, and are usually used for liquid samples volumes of between 10 ul to 1,500 ul. Microscope slides are utilized for tissues samples and very high density arrays of tiny samples that can be bound to the surface of the slide. Microtiter plates are built like arrays of small microfuge tubes, and are available in a multitude of formats with varying materials, well geometries and sample densities, but all share the same basic footprint and they are typically used for liquid samples that are between 10 ul and 1,500 ul in volume.

Because the microfuge tube offers relatively high volume of reactants and low number of biological samples, the trend for clinical diagnostics, industrial microbial detection, and pharmaceutical and academic research has been to reduce the reaction volume and increase the throughput of these processes. To this end, higher density microtiter plates and slide-based microarrays have become more commonly used. These formats are of particular interest because they offer the ability to perform parallel experiments, reduce reagent consumption, and utilize smaller, relatively less expensive laboratory and analytical instrumentation.

The vast majority of microtiter plates in use conform to a set of standards codified by the Society for Biomolecular Screening (SBS) over the last decade. The plates typically have 6, 24, 96, 384 or even 1536 sample wells arranged in a 2:3 rectangular matrix. For thermal cycling applications, 96-well and 384-well formats are, by far, the most commonly used. 96-well microtiter plates typically consist of an 8×12 array of wells of 9 mm center-to-center pitch and an inner diameter of 5.5 mm. Depending on the variety of plate, each well can hold a maximum of between 100 ul and 200 ul of reaction volume. 384-well plates, halve the spacing, such that the plates now offer a 16×32 format, with 4.5 mm pitch, 3.0 mm inner diameters, and maximum sample volumes of 40 ul to 60 ul. The geometries of the wells vary depending upon the application—from square-shaped wells with flat bottoms to round wells with conical bottoms. Most biological chemistries performed in a microtiter plate are solution-based, but surfaced based chemistries can also be performed.

One of the unique requirements of microtiter plates designed for thermal cycling applications are the protrusion of conical shaped tubes below the bottom surface of the plate. These “cones” seat snuggly into matching metal receptacles that are heated and cooled alternately. The interface allows for the efficient heating and cooling of samples because the ratio of surface to volume increases dramatically, as compared with flat bottom plates in which the heat conduction only occurs via the bottom surface of the well. Additionally, the samples are heated uniformly in Z-dimension, which is in contrast to flat-bottom plates, which when heated from only the bottom cause a gradient of temperature from the bottom to the top of the sample within the well.

To date there has been little progress made in designing manufacturing and selling microtiter plates of densities below a 4.5 mm pitch for thermal cycling purposes. This is in contrast to the high throughput screening fields in which 1536-well microtiter plates are routinely used for ELISA and ligand-binding analyses. These 1536-well plates offer very small wells on a 2.25 mm pitch, with volume capacities between 1 ul and 20 ul. Such plates are typically made of polystyrene and are designed with a flat bottom for optimal optical characteristics and ease of manufacture. Although such plates are optimal for performing biological reactions at or near room temperature, they are not designed to handle the stresses and thermal performance needs of high temperature cycling applications such as PCR.

As the well density of microtiter plates have increased, regardless of the application, the need for handling said samples in an automated fashion has been a requirement for such plates. Thus, it has been imperative to design plates that are stable throughout the processing of the biological samples so that such samples can be dispensed or removed reliably and accurately. Paramount to such precise liquid handling is the dimensional stability of the plate, even under high temperature applications. For 384-well microtiter plates special materials and/or specialized features have been used to retain this stability. To date, however, these same types of features and materials have been unable to be transferred to the higher well density used, for example, in 1536-well plates for thermal cycling applications.

U.S. Pat. No. 6,340,589 discloses a microtiter plate which is comprised of two separate parts formed of different materials. Wells are contained in the upper part (deck portion) of the plate and is supported by the lower part (skirt portion). Instead of a complete deck, the upper part may include a meshwork of links, which connect the wells at their upper ends to each other. A major drawback of such a plate is that it has to be manufactured in several steps and the parts need to be attached together before use. Moreover, the rigidity of the upper portion, and consequently the whole plate is low due to the structural non-integrity.

U.S. Pat. No. 5,922,266 discloses an injection molding method and apparatus, which may be modified to suit for producing the plates according to the present invention. As such, the document concerns manufacturing of optical devices, such as lenses.

### SUMMARY OF THE INVENTION

It is an aim of the invention to provide a microtiter plate, which enables increasing the throughput of multiple-sample high temperature thermal cycling processes. In particular, it is an aim of the invention to provide a novel thermally stable and robust construction especially for use for microtiter plates having a well-to-well pitch as small as 2.25 mm, and even less, and a wall thickness of less than about 0.0065 inch (less than about 0.17 mm).

It is also aim of the invention to provide a novel kind of microtiter plate, which has thermal performance characteristics superior to known plates.

It is also an aim of the invention to provide a novel method for producing a microtiter plate, which enables manufacturing of a dense grid of sample wells having an ultra-thin wall thickness for improved thermal performance.

The invention is based on the idea of providing a microtiter plate having a plurality of wells in the form of a grid and further providing ribs between the walls of the wells. The ribs are typically provided in two dimensions such that they connect each well to two, three or four adjacent wells, depending on the position of the well in the grid. The ribs lie generally in a plane perpendicular to the plane of the grid defining the well positions. In such a manner the structure of the plate can be reinforced so as to still allow for considerable portion of the wall of each well to contact a sample holder so that efficient heat transfer to the sample within well can occur.

According to a preferred embodiment, the well walls at the open ends of the wells (i.e. rims of the wells) are shared between adjacent wells. According to a still further embodiment, the wells are otherwise round-shaped in cross section, but the interiors of the wells are shaped roughly rectangular in cross section at their open ends in order to achieve higher density of wells in the grid but still maintaining high internal volume of the wells.

In the method according to the invention, a microtiter plate comprising a plurality of thin-walled wells in the form of a grid is produced by injection molding by injecting molten mold material to an oversized injection mold comprising several well-forming cavities having an initial volume, at least one of the well-forming cavities being connected to at least one other well-forming cavity by a planar flow channel having a general direction perpendicular to the plane of the well grid, the method comprising a step of reducing the volume of the mold for displacing said mold material in the well-forming cavities and in the flow channels in order to produce a thermally stable microtiter plate having at least one of the wells connected to at least one another well by a rib. Flow channels may be provided so as to connect each of the wells to at least one another well, preferably to two, three or four neighboring wells in the grid by ribs.

More specifically, the microtiter plate according to the invention is characterized by what is stated in claim 1.

The method according to the invention is mainly characterized by what is stated in claim 17.

The kit is characterized by what is stated in claim 27.

Considerable advantages are obtained by means of the invention. When the structure of the plate is concerned, the ribs provide support for the wells, which can therefore be designed thin-walled and placed in a dense grid. Thus, the ribbed structure enables manufacturing of plates, wherein ratio of the density of tubes to the wall thickness of the tubes is fundamentally increased in relation to prior plates.

By additionally sharing portions of the walls between the wells, a desired combination of high well density together with high thermal transfer which is imparted by the ability to surround the well with the thermal control source (sample block of a thermal cycler) and good mechanical stability/rigidity is achieved. That is, sharing allows for
a mechanically robust interconnection of the wells due to maximized contact area at the joining point and,
the upper surface of the well possessing a geometry and surface topography for effective closure and sealing of the wells during thermal cycling using commercially available sealing films or elastomer sealing pads.
   The above applies, in particular, to otherwise round but square-shaped at the upper end geometry of the wells.

Conventional molding techniques allow only tube walls roughly greater than 0.01″ (0.26 mm) due to untimely “freezing off” of the resin as it flows through the thin areas, that is, it is hardened before the part can be fully formed and packed out. Even if one could tolerate such a thick tube wall, conventional mold design would necessitate injection of the resin from one side of the part and venting on the opposing side of the part, perhaps at the mold parting line or at the tip of the tube(s). Nevertheless, if the tubes had no connecting fins (ribs), as in the present invention, the resin would likely have to be injected at each intersection of walls. This could likely be accomplished but would result in a very high density of gates and a less than optimal mold design.

In other words, the ribs make the molding of small-sized protrusions by injection molding possible in an advantageous manner, as they allow the proper flow of resin in a plastic-injection molding of the wells without the requirement for an unduly high number of air-vents in the mold. That is, the injection mold may comprise a plurality of air-vents such that the number of air-vents is considerably smaller, typically at least 20%, preferably at least 50% smaller, than the number of wells in the microtiter plate. In the present process, a defined volume of resin sufficient to form the part be injected into the part forming cavity while it is held partly open and then the cavity be closed, thereby compressing the resin such that it fills the cavity and forms the thin wall sections desired. As the compression takes place the air and volatiles (gas) in the voids must be evacuated through vents. Since the polymer fronts advance toward the exterior of the part during both injection and compression, the ribs facilitate flow of gas toward the vents by providing a connecting path through which the polymer resin may flow. Using conventional mold designs, it has proven to be very difficult to mold tightly-spaced wells without creating a number of air-vents equal to the number of wells, which however in this high density well format, is not practical.

In order to maximize thermal performance a plate must be designed with conical wells in a material suitable for biological reactions. Such a design must take into account thermal transfer characteristics, ability of the plate to handle thermal stresses from cycling and the ability to add and remove biological samples using an automated liquid handler. The present invention is particularly advantageous, when a plate having a dense grid of tubes having a wall thickness of less than 0.0065″ (0.17 mm) is desired. By a dense grid, we mean a grid having a well-to-well spacing (pitch) of 2.25 mm or less. Incorporation of conical tubes in such a high density has not been previously possible, but the design elements associated with the plate and disclosed in this document and the use of a novel plastic-injection molding process allows for the creation of a plate that is both manufacturable and allows for ultra-thin, conical walls for efficient transfer of heat to the samples. The term “ultra-thin” is considered to cover at least the thickness range extending from 0.0025″ to 0.0065″.

In regards to performing common molecular biological reactions requiring high temperature thermal cycling, the ribbed-conical well format allows for the potential of:
i) high sample density of wells (2.25 mm pitch and less),
ii) optimized thermal transfer of heat,
iii) easy dispensing of low sample volumes,
iv) easy sample recovery at bottom of conical tube, and
v) lower reagent usage.

Forming the plate further into a reduced-size format (e.g., a slide-sized format having a footprint of roughly ¼ than that of a standard microtiter plate), as described in detail later in this document further allows for:
-minimization of warping shrinkage of the plastic plate, and
the creation of smaller, less expensive instrumentation to perform biological assays, than are afforded by standard microtiter-sized plates of lower density.

As mentioned above, the shape of the wells is preferably conical. That is, at least the lowermost part of the well is tapering towards the bottom of the well (the wells are most advantageously “v-bottomed”). Heat transfer between the sample within such a conical well and the heating/cooling receptacle is efficient because:
i) the conical geometry has a relatively high surface to volume ratio,
ii) the walls can be molded in such a fashion that the thickness is even ½ that of conventional tubes, thus reducing impedance to thermal conductance caused by the plastic, and
iii) heating along the entire height of the sample allows for uniform temperatures from top to bottom—important for enzyme kinetics of the reaction.

Small wells (in particular, those having inner diameters of less than 2 mm) provide lower reagent usage, because smaller wells have less surface area (and head space) to lose sample volume via vapor pressure. Also dispensing and retrieval of samples is made more reliable and repeatable because the wells themselves are cone-shaped allowing small volumes to have enough Z-dimension aspect to allow a pipette tip to operate properly. In addition, the stress-free molding process of the plates coupled with a frame assembly (detailed below) allows for small volumes to be retrieved with precision.

By “adjacent” or “neighboring” wells, we mean neighboring wells either in the principal grid directions or in the diagonal directions of the grid.

The embodiments of the invention are examined more closely below with reference to the attached drawings.

### BRIEF DESCRIPTION OF THE DRAWINGS

FIG. 1 shows a partial bottom view of a ribbed microtiter plate according to one embodiment of the invention.

FIG. 2 shows a partial perspective view of the ribbed microtiter plate of FIG. 1.

FIG. 3 shows a partial side view of the ribbed microtiter plate of FIGS. 1 and 2.

FIG. 4 shows a perspective view of the ribbed microtiter plate of the previous Figures (only four first well rows shown).

FIG. 5 shows a partial top view of a ribbed microtiter plate of the previous Figures.

FIG. 6 shows a partial cross-sectional view of a microtiter plate between mold members during the molding process.

FIG. 7 shows a first perspective view of an exemplary sample tray assembly which can be used in order to house ribbed microtiter plates.

FIG. 8 shows a second perspective view of the sample tray assembly of FIG. 7.

### DETAILED DESCRIPTION OF EMBODIMENTS

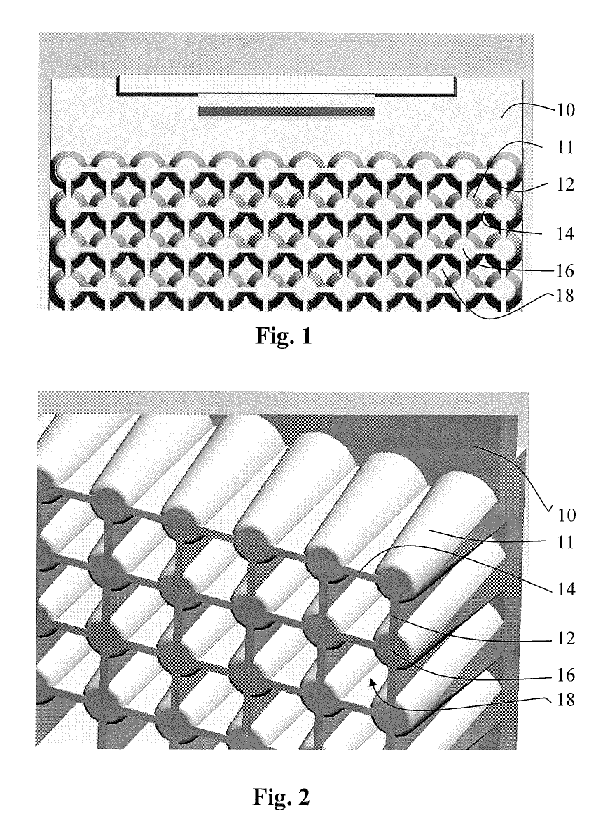

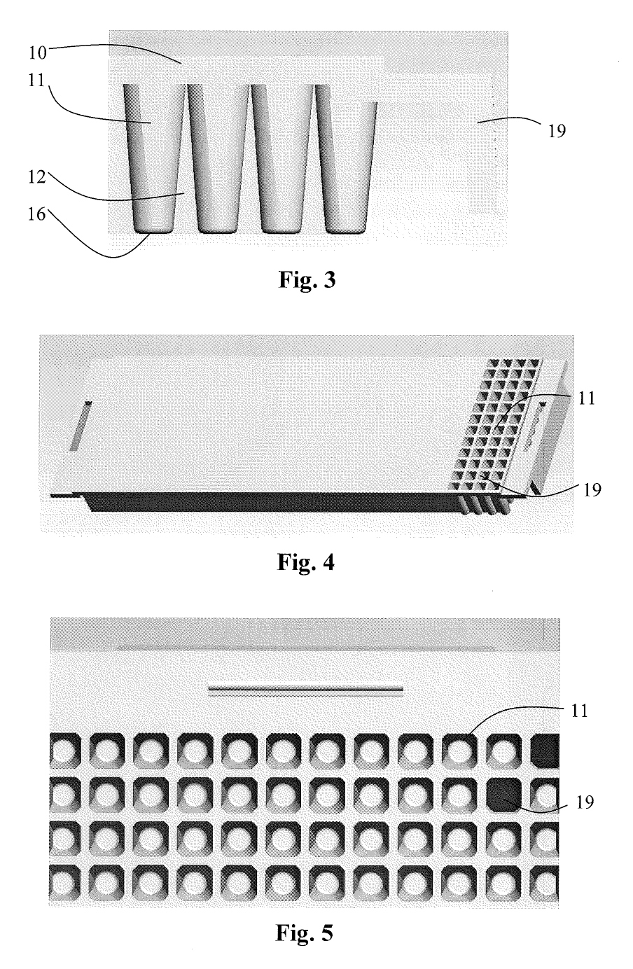

FIGS. 1 to 5 show one embodiment of a microtiter plate having round conical wells 11. The deck of the plate is denoted with a reference numeral 10. The wells 11 protrude in a parallel manner from the deck 10 such that their upper (open) ends abut on the upper surface of the deck 10. There are two set of ribs present: the first ribs 14 are arranged to connect the wells in first direction (in the direction of well rows) and the second ribs 12 are arranged to connect the wells in a second direction (in the direction of well columns). In this example, the ribs protrude all the way from the bottom of the deck to the level of the bottoms 16 (closed ends) of the wells, and connect to each other at the bottoms 16 of the wells. An interstitial space 18 bordering on the bottom of the deck and four wells and four respective ribs is formed in the middle of each neighboring four wells set. As the wells 11 have the shape of a truncated cone and the ribs are connected from their sides to the wells of the wells 11 in full length, each of the ribs has a triangular shape.

FIGS. 4 and 5 show the upper surface of the plate. Due to the high density and conical shape of the wells, the interiors of the wells are shaped roughly rectangular in cross-section at their upper ends. This is to prevent overlap of adjacent well cavities and to allow decent sealing of the plate. Thus, extra material is provided at the upper ends of the well walls order to separate the otherwise round-shaped wells from each other. A biological sample 19 is provided in some of the wells.

In the most preferred embodiment of the invention, the vessel possesses a plurality of wells having walls consisting of inner and outer surfaces, said wells being generally independent and conical in nature but transitioning, at their open ends, to a more square geometry at which point they interconnect by means of shared wall surfaces at said open ends. The upper surface of the vessel is defined as that which coincides with the open ends of the tubes and whose uppermost surface plane is defined by the ends of open tube.

The closed ends of the essentially conical tubes, being the furthermost point from the open end of the tubes, define the lower surface plane of the vessel. When viewed form the bottom, or lower surface plane, it is obvious that, due to the essentially tapered/conical geometry of the array of individual wells, an array of openings having a female orientation remains between the individual wells. This array of openings allows for the vessel to mate intimately with a corresponding array of male features formed in the thermal control block of the thermal cycling instrument. Since the openings present on the underside of the vessel extend nearly to the upper surface of the vessel, it becomes possible to surround the sample containing area of the well of the vessel nearly completely with the thermal control source, a necessary feature for effective performance of vessels intended for PCR applications.

In order to improve mechanical rigidity of the vessel, a thin standing wall, i.e., rib, is introduced between the exterior of each well. Said wall extends from the underside of the vessels upper surface to a point corresponding to the tip of the well's closed end. Each wall is aligned with, and parallel to, the centerline of each well and extends perpendicularly in each direction thereby interconnecting each well with it's neighboring well. This construction, while retaining the important feature of open space surrounding each well, allows for extraordinary rigidity and stability of the vessel.

According to one embodiment of the present molding process, a plate is produced by delivering plasticized resin into a mold cavity sufficiently to fill the cavity and evenly displacing a portion of that resin within the cavity by compressing the resin by walls of the cavity, typically by clamping with core pins which form the internal diameter (ID) of the sample tube, to form the desired wall thickness. The resin is then allowed to cool in the pressurized cavity thereby forming a thin-walled vessel having the desired uniform shape and reduced internal stresses. In the clamping phase, the resin fills evenly the mold cavity, including the well wall portions, rib portions, and a deck portion usually present for binding the upper ends of the tubes firmly to each other. The deck portion could also be left out, because binding of the tubes to each other can be achieved by the ribs, in particular when reinforced from their upper ends by sharing the walls of adjacent wells, as described above.

A detailed description of a process suitable for the purposes of this document is given in the still unpublished patent application PCT/IB2006/002452, which discloses one method of producing thin-walled microtiter plates and is incorporated herein by reference. The method suits best for producing relatively sparse plates, but can be applied to the concept of the present invention in order to obtain additional advantages, as described in detail herein.

According to a preferred embodiment of the present method, the phases of injection of the molten material and clamping are carried out successively in order to secure as homogeneous structure of the thin-walled vessel as possible. This is contrary to the teachings of U.S. Pat. No. 5,922,266, where the injection and compression is carried out simultaneously. In addition, U.S. Pat. No. 5,922,266 does not include any teachings about using the method for producing thin object portions and, in particular, for producing robust vessels for thermal cycling applications.

Closing of the core pins does two things. Firstly, it compresses the tube walls to the desired thickness, and, secondly, evenly displaces the polymer in the mold cavity to produce an equalized packing force on the part prior to cooling. In traditional injection molding techniques, the tubes must be either filled or vented at each tube in order to flow material such that it will completely fill the tubes. This, however, makes the molding process unduly complex. The present invention allows for filling and/or venting of the tubes at the region of the ribs, whereby reduction of filling/venting points is possible and no undesired molding residues are produced in the tube walls.

In more detail, the method comprises in carried out by injection molding in an injection molding machine using a molten thermoplastic resin, and comprises the steps of:
forming an oversized mold cavity with an opposing pair of mold members of the injection molding machine, the mold members being movable relative to each other and between which mold members the sample tube is formed;
injecting into said oversized cavities a volume of resin exceeding the prescribed volume of the sample tube to be formed; and
applying force to the mold members in order to reduce the volume of the mold cavity for displacing molten polymer in the cavity and for compressing the polymer to form the sample tube.

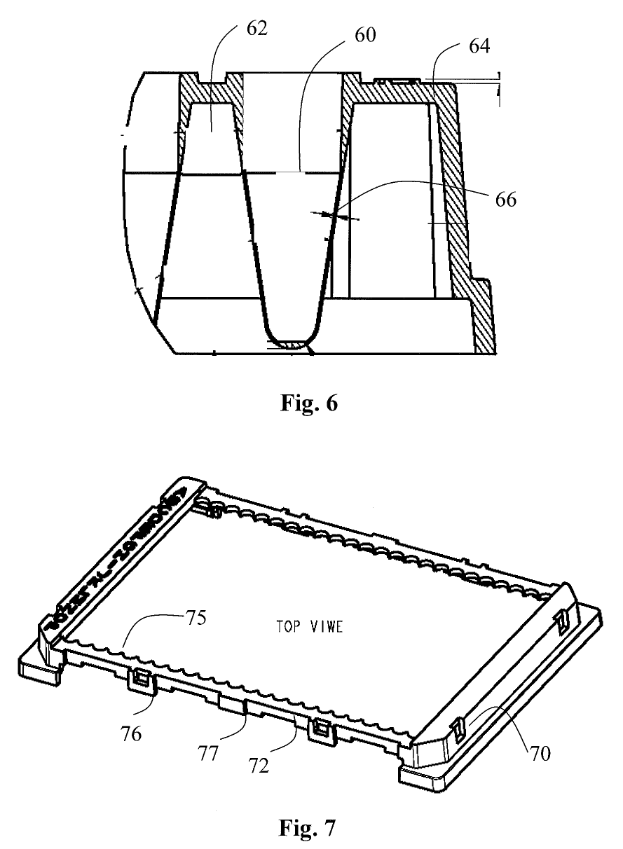

FIG. 6 shows a vessel 64 clamped between mold members. The upper mold member comprises core pins 60, which define the internal diameter (ID) and the internal shape of the wells. The lower mould member 62 defines the outer diameter (OD) and shape of the wells, and the shape of the ribs. The thin wall portion of the wells is denoted with reference numeral 66. In FIG. 6, the image plane of the cross-section lies slightly off the plane of the ribs in order to show the protrusion of the lower mold member to the interstitial space between the wells more clearly.

The process according to the invention allows for increases in the density of wells with much thinner walls associated with the conical bottom portions of the wells. A wall thickness of less than about 0.0065 inch (less than about 0.17 mm) at the bottom portions of the wells in combination with a small (less or equal than 2.25 mm) well pitch can be achieved, still maintaining the robustness of the plate due to relieved stresses and small variations in the wall thickness. The thinner well walls maximize heat transfer such that high rates of thermal transfer can occur, allowing for overall shorter assay times and higher sample processing rates. The rate at which a sample is heated and cooled during a conventional thermal cycling reaction may account for up to 50% of the total assay time, whereby halving the wall thickness enables reduction of the total assay time by as much as 25%. The thicker walls and other structures associated with the tops and sides of the plates provide additional rigidity and structural integrity to the entire plate. These features will help minimize the shrinking and warping of the plate after repeated exposure to hot and cold temperatures. Minimization of shrinking and warping both before and after thermal cycling is a requirement for automated liquid handlers to repeatedly and reliably dispense or aspirate small volumes of sample at the bottoms of the tube.

As an advantageous practical embodiment of the present invention, a microtiter plate format is introduced, which comprises in combination:
a plate comprising a plurality of wells supported by ribs as disclosed above and arranged in a grid having a predetermined pitch,
a number of wells in a first dimension of the plate, which corresponds to the number of wells in a first dimension of an SBS standard plate and the number of wells in a second dimension of the plate, which corresponds to a fraction of the number of wells in a second dimension of an SBS standard plate.

Hence, such a plate can be designed, for example, in a quarter-sized format of a standard microtiter plate (roughly corresponding to the size of a microscope slide-format). The smaller footprint of the plate further reduces the dimensional stresses of the plate so that warping of the plate as it is ejected from an injection molding machine is minimized. This feature also reduces problems associated with flow dynamics of molten plastic as it fills the cavities of the mold, such that the cavities are more likely to fill at the proper pressures.

Building smaller, less expensive instrumentation is a function of the smaller size of the plate. An example might be a thermal cycler designed for the smaller, slide-sized plate format. Such an instrument would have lower power consumption because only ¼ of the standard microtiter plate area needs to be heated and cooled. Also, related the heat sink for the thermally conductive sample holder could also be up to ¼ the size because of the plate format and lower power usage. Both a smaller power supply and smaller heat sink could translate to a significantly smaller system (as the power supply and heat sink may contribute as much as 50% of the instrument volume requirements).

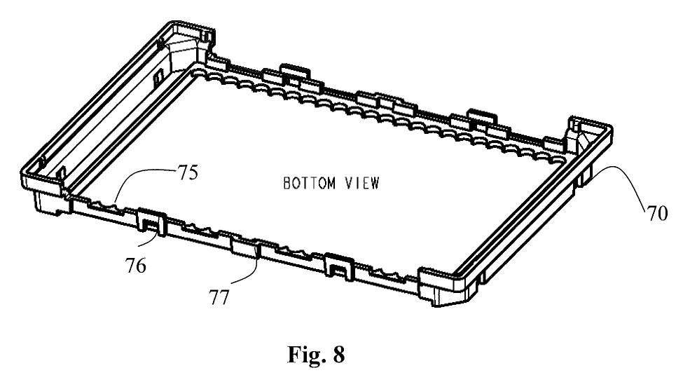

With reference to FIGS. 7 and 8, according to one embodiment, there is provided a tray assembly, which is capable of receiving and holding a plurality of reduced-sized plates. Such tray assembly generally comprises a frame 77 having two parallel first frame elements 70 and two parallel second frame elements 72, the frame elements being perpendicularly connected to each other to form a generally rectangularly shaped frame, the inner edges 75 of the frame elements defining a central opening and the frame being capable of accommodating and immobilizing a plurality of adjacent sample plates such that their sample wells at least partially protrude through the central opening of the frame. Preferably, the outer peripheral dimensions of the frame meet the SBS standards, whereby the present sample plate assembly can be used for processing of biological samples in, e.g., thermal cyclers, which are conventionally operating on SBS standard microtiter plates. A detailed description of a tray assembly suitable for the purposes of this document is given in the still unpublished patent application PCT/FI2006/050379, which is incorporated herein by reference.

FIGS. 7 and 8 show and example of tray designed for a 4×96 well plate configuration, but a similar tray may also be manufactured for a 4×384 well plate configuration in order to fit together with the most preferred form of the plates according to the invention. Needless to say, 2×768, 3×512, 6×256 etc. configurations, and all other configurations in which plates can be fitted side-by-side in order to fill a rectangular frame are possible, and may have advantages in some applications.

The described frame design combined with the ribbed-well design further helps to accomplish the goals of the invention, and to maintain robust manufacturability and manageability of the plates. Reduced-sized plates can be assembled, side-by-side, on a microtiter-sized frame to allow the manipulation of these plates by standard liquid handling and robotic workstations commonly used in life science research. Thus, the ability for two or more, typically four, of these slide-sized plates to be combined into one microtiter-sized tray assembly still maintains some of the key advantages of microtiter-sized plates such as: i) use of standard liquid handling devices, and ii) compatibility with existing laboratory and analytical instrumentation. Most semi-automated and fully automated liquid handlers for molecular biological reactions remove and dispense liquid as either a single tip, a row of 4, 8, or 12 tips, or an array of 96 or 384 tips (in a 8×12 or 16×24 tip array respectively). Such liquid manipulating instruments, are designed to hold a standard, SBS-compatible, microtiter plate in a position relative to the dispensing tips and either move the tips, or the plate (or both) to address the appropriate wells. The key to maintaining the compatibility is to offer a format of correct X-Y dimensions, and a correct well-to-well spacing. Like with liquid handling devices, common types of laboratory equipment and analytical instrumentation have been designed to work specifically with microtiter plates of particular X-Y dimensions and well-to-well spacing.

An exemplary, yet preferred, plate format is based on a slide-sized plate concept with 384 conical wells protruding from the bottom surface of the deck of the plate. The 384-well slide-sized plate preferably has a format of 12×32, with a center-to-center pitch for adjacent wells of 2.25 mm. The maximum sample volume will be between 10 ul and 20 ul. The plate can be sealed by any of the following methods which will allow for efficient sealing to as low as 1 ul reaction volume with the application of pressure from the top: i) heat-sealing films, ii) pressure sealing films, and iii) reusable sealing mats. The wells are designed to allow for efficient heat transfer of samples and removal of low reaction volumes with standard pipeting tools. The material of the plates will be of polypropylene, or like material, that offers good thermal conductivity, hydrophobicity and low interference with molecular biological reactions.

Paramount to good manufacturability and rigidity of the plate, ribs connect the sides of the wall of each conical well. The ribs can be in any of a number of different configurations, but the preferred embodiment is to have the ribs arranged in a standard square grid configuration with the sides of each square equivalent to the pitch used and the intersection of the four ribs will meet at the bottom of each well. Moreover, the height of each rib will be defined as starting at the bottom surface of the plate deck and stretch at least halfway down the well depth axis, preferably all the way to the bottom of each well, thus making an “egg crate” appearance to the bottom of the plate. The thickness of each rib can be optimized for proper flow of resin in the mold, and maximized exposed surface area of the tube wall to contact the heating/cooling receptacle. A typical thickness of a rib is between 0.008 and 0.020 inches as measured at the lower surface of the rib. The walls of the wells, at points in which the ribs are not joined, will be of a thickness of less than 6/1000ths of inch.

Ribs are typically of generally planar form and lie perpendicularly to the upper plane of the vessel. They may, however, exhibit a gently sloping (tapering) or patterned form. Ribs may also be provided in configurations not explained here in detail, for example, in oblique manner (diagonally in the grid from well to well). In that case, the ribs connecting four wells in a square-like vertices of the well grid would form an X-shaped interconnecting structure. A multi-facetedly (i.e., between nearest neighbors and between diagonal neighbors) ribbed structure would even further add to the rigidity of the product, however, at the expense of usable heat transfer area. In the case of large plates (e.g., standard-sized high-density plates), this may, however, be beneficial.

The conical wells themselves preferably have an inner draft angle of between 3° and up to 10°. The cones protrude between 4.0 mm and 7.0 mm from the bottom of the deck. The tubes thicken gradually from bottom to top such that the thinnest portion of less than or equal to 6/1000ths of inch will be maintained at all points in direct contact with the heating/cooling receptacle and increase thereafter to give the wells added strength. The rims of the wells are preferably shared between wells. Regardless of the configuration the rims preferably have a curvature along the top surface so that pressure-based sealing films will form a vapor-tight contact along the entire periphery of the well rim.

Four 384-well slide-sized plates will be capable of mating with a rigid frame so that the complete assembly resembles closely a standard microtiter-sized plate. The overall format of the mated frame/plate assemblies will be 32×48 wells (equivalent to a 1,536-well microtiter plate). The frame itself will be of SBS standards, and made of a material that is both rigid and heat-resistant, so that it holds the slide-sized plates in a regular and repeatable position, even after stresses caused by standard laboratory processes and conditions. The addition or removal of a plate, or series of plates from the frame assembly can be accomplished manually, without the aid of tools, or alternatively can be incorporated into a robotic system, which will perform such tasks in an automated fashion.

The mated frame/plate assembly will be compatible with general laboratory equipment and analytical instrumentation. Such general lab equipment includes centrifuges adapted to spin individual and stacked microtiter plates; thermal cyclers that accommodate v-bottom microtiter plates; simple heaters and chillers that accept microtiter plates; and liquid handlers that are designed to manipulate reactions in wells configured within a microtiter plate format. Examples of analytical instrumentation that will accept microtiter-sized plates are DNA automated sequencing systems, florescence and colorimetric plate readers, and real-time, quantitative PCR instruments.

The described frame/plate assembly provides a convenient way of achieving an ultra thin walled densely designed vessel for increased thermal performance and sample throughput. However, a 1,536-well plate can also be manufactured as a single piece by means of the described process utilizing ribs between the tubes.

In one embodiment, the vessel is provided with an integral deck part having an upper surface facing to the direction of the open ends of the wells and a lower surface facing to the direction of the closed ends of the wells and being connected to the well walls in the vicinity of the open ends of wells. This applies in particular to an embodiment, where the rims of the wells are separate and raised above the deck surface. In that case the ribs may be connected to the lower surface of the deck part. However, as described above and in FIGS. 1-5, in a preferred embodiment the walls of the wells are shared between the wells at their upper ends in order to provide a more dense grid, whereby the deck in these shared areas is inherently formed of the rims of the wells and usually has no distinguishable lower surface at the locations where the walls of the adjacent wells meet.

Having read the description above, it is apparent to a person skilled in the art that the plate preferably consists of a single and structurally integral unit made from a biocompatible material. The material of the plate is most advantageously suitable for the temperature range of PCR processes.

The exemplary embodiments described above and in the appended claims can be freely combined within the spirit of the invention.

1. A vessel for thermal cycling applications comprising, in the form of a planar two dimensional grid having a predefined pitch, a plurality of sample wells each having a well wall, which defines an open well end and a closed well end, wherein there are provided a plurality of ribs between pairs of adjacent wells, the ribs being connected to the walls of the wells and extending essentially in a plane perpendicular to the plane of said grid for reinforcing the structure of the vessel.

2. A vessel according to claim 1, wherein the wall of each of the sample wells is connected to the walls of at least two, typically two, three or four, depending on the location of the well in said grid, adjacent sample wells by ribs.

3. A vessel according to claim 1 or 2, wherein the ribs are provided in a square grid configuration, the sides of each square having a length equivalent to the pitch used and the ribs intersecting at the closed end of each well.

4. A vessel according to claim 1, wherein the well walls at the open ends of the wells are shared between adjacent wells.

5. A vessel according to claim 1, wherein the wells are generally round-shaped in cross section.

6. A vessel according to claim 1, wherein the interiors of the wells are shaped roughly rectangular in cross section at their open ends in order to achieve higher density of wells in the grid.

7. A vessel according to claim 1, wherein the ribs are triangular in shape.

8. A vessel according to claim 1, wherein the ribs extend from the vicinity of the open ends of the wells at least halfway down the well depth axis, preferably down to the bottom level of the wells.

9. A vessel according to claim 1, wherein said pitch is 2.25 mm or less.

10. A vessel according to claim 1, wherein each of the well walls is provided with a thin wall portion which has a consistent wall thickness of less than about 0.0065 inch (less than about 0.17 mm).

11. A vessel according to claim 1, wherein the vessel is made of thermoplastic material, such as polymeric resin, which has been hardened in pressurized condition, the pressurized condition being achieved at least partly by mechanical clamping of molten material.

12. A vessel according to claim 1, wherein the number of wells in a first dimension of the vessel corresponds to the number of wells in a first dimension of an SBS standard plate and the number of wells in a second dimension of the vessel corresponds to a fraction of the number of wells in a second dimension of an SBS standard plate.

13. A vessel according to claim 12, wherein said fraction equals a quarter of said number of wells in the second dimension of the SBS standard plate.

14. A vessel according to claim 1, wherein the vessel has an outer form adapted to allow placing two such vessels side-by-side such that the well-to-well spacing over the contact region of the plates equals said pitch for enabling several such vessels to be used in forming a larger geometrically compatible vessel.

15. A vessel according to claim 1, wherein the wells are conical, preferably having the form of a truncated cone.

16. A vessel according to claim 1, which consists of a single structurally integral unit made from material suitable for biological reactions taking place in the vessel.

17. A method of manufacturing a sample vessel by injection molding, the vessel comprising a plurality of sample wells in the form of a planar two dimensional grid having a predefined pitch, comprising:
injecting molten mold material to an oversized injection mold cavity comprising several well-forming cavities having an initial volume and being arranged in a grid, each of the well-forming cavities being connected to one adjacent well-forming cavity by a planar flow channel extending essentially in a plane perpendicular to the plane of said grid, and
reducing the volume of the well-forming cavities for displacing said mold material in the cavities and in the flow channels in order to produce a vessel having each of the wells connected to at least one another well by a rib.

18. A method according to claim 17, wherein each of the well-forming cavities is connected to at least two, typically two, three or four depending on the location of the well-forming cavity in said grid, adjacent well-forming cavities such a planar flow channel.

19. A method according to claim 17 or 18, wherein the mold material is thermoplastic resin, such as polypropylene.

20. A method according to claim 17, wherein the mold material is allowed to cool in a pressurized mold cavity for preventing deformations and internal stresses of the vessel.

21. The method according to claim 17, wherein the step of reducing the volume of the well-forming cavities comprises reducing the volume as much as is required to produce wells having a wall thickness at some part of the well walls consistently less than about 0.0065 inch (0.17 mm).

22. The method according to claim 17, wherein the flow channels are provided in a square grid configuration, the sides of each square having a length equivalent to the pitch used and the intersections of the flow channels taking place at the bottom of each well.

23. The method according to claim 17, wherein said pitch is 2.25 mm or less.

24. The method according to claim 17, wherein the mold cavity comprises several venting points, the number of which is smaller, preferably at least 50% smaller, than the number of said well-forming cavities.

25. The method according to claim 17, which is performed with an injection molding machine and comprises the steps of:
forming an oversized mold cavity with an opposing pair of mold members of said injection molding machine, the mold members being movable relative to each other and between which mold members the sample wells are formed;
injecting into said oversized cavity a volume of resin exceeding the prescribed volume of the sample wells to be formed; and
applying force to said mold members in order to reduce the volume of the mold cavity for displacing molten polymer in the cavity and for compressing the polymer so as to form the vessel.

26. A vessel produced according to the method of claim 17.

27. A kit for processing biological samples comprising a tray assembly and a plurality of sample plates designed to fit into the tray assembly, wherein
the tray assembly comprises a generally rectangular frame having perpendicularly connected frame elements defining a central plate receiving portion having a width and a length, whereby said tray assembly is capable of accommodating the sample plates side by side in the plate receiving portion; and
the sample plates comprise vessels according to claim 1.

28. A kit according to claim 27, wherein the plate receiving portion comprises a central opening or central recess.

29. A kit according to claim 27, wherein the tray assembly and the sample plates comprise mounting means for assisting positioning and immobilizing of the sample plates in the frame.
